(19) United States
(12) Patent Application Publication
(10) Pub. No.: US 2024/0115184 A1
(43) Pub. Date: Apr. 11, 2024

(54) METHOD FOR PREDICTING CHRONIC DISEASE ON BASIS OF ELECTROCARDIOGRAM SIGNAL

(71) Applicant: VUNO Inc., Seoul (KR)

(72) Inventors: Youngjae SONG, Suwon-si (KR); Woong BAE, Seoul (KR)

(21) Appl. No.: 18/263,102

(22) PCT Filed: Jan. 27, 2022
(86) PCT No.: PCT/KR2022/001497

§ 371 Date: Jul. 26, 2023

(30) Foreign Application Priority Data
Jan. 27, 2021 (KR) 10-2021-0011187

(51) Int. Cl. A61B 5/327; A61B 5/00; A61B 5/364; G06N 20/00; G16H 50/30

(52) U.S. Cl. CPC A61B 5/327; A61B 5/364; A61B 5/7239; A61B 5/7275; G06N 20/00; G16H 50/30

(57) ABSTRACT

According to an embodiment of the present disclosure, disclosed is a method for predicting a chronic disease based on an ECG signal performed by a computing device. The method may include generating lead-specific integrated data based on the ECG signal. The method may include generating N-dimensional input data based on the lead-specific integrated data. The method may include predicting the chronic disease based on the N-dimensional input data. The method may include generating prediction information on the chronic disease to be provided to a user.

### BACKGROUND

### Technical Field

The present disclosure relates to a method for analyzing a bio signal, and more particularly, to a method for predicting a chronic disease based on an electrocardiogram signal using machine learning.

### Description of the Related Art

Development of technologies that use electrocardiogram signals as basis data for diagnosis and prediction of chronic diseases are being attempted in various ways. One of them is a scheme of utilizing artificial intelligence. In order to utilize the artificial intelligence, it is necessary to precede a task of processing the electrocardiogram signal to be interpreted by the artificial intelligence. When a one-dimensional ECG signal itself is used as an input for an artificial intelligence model, information on a lead from which the ECG signal is measured is included in a computation channel, so there is a problem of lowering the degree of freedom for processing a correlation between leads that should be considered for diagnosis and prediction of the chronic disease. Therefore, it is necessary to adjust an input form of the ECG signal, and to process the ECG signal so as not to lose unique information in the process of adjusting the input form.

According to the above-described necessity, conventionally, the one-dimensional ECG signal is processed into a two-dimensional image form through frequency domain conversion and used as the input of the artificial intelligence model.

Korean Patent Registration No. 10-2119169 (May 29, 2020) discloses a two-dimensional image generation method of an ECG signal.

### BRIEF SUMMARY

### Technical Problem

The inventors have realized that various conventional methods have a problem in that interpretation of time-series information becomes difficult. Further, conventionally, attempts have been made to separately input lead information in order to use the one-dimensional signal itself. The inventors have realized that the attempts also have a problem that makes it difficult for domain experts to interpret the importance of each lead for diagnosing the chronic disease, even though the AI model can utilize a correlation between leads in a computation process. The present disclosure is contrived to respond to the background art, and has been made in an effort to provide a method for predicting a chronic disease based on machine learning, which is configured to interpret a mutual importance between leads for ECG measurement without losing unique information of an ECG signal.

### Technical Solution

In order to realize the object, according to an embodiment of the present disclosure, disclosed is a method for predicting a chronic disease based on an ECG signal performed by a computing device. The method may include: generating waveform-specific interval information and gradient information of an ECG signal from ECG data; generating lead-specific integrated data based on at least one of the ECG data, the gradient information or the waveform-specific interval information; and generating N-dimensional input data based on the lead-specific integrated data.

In an alternative embodiment, the generating of the gradient information and the waveform-specific interval information may include generating ECG data by sampling the ECG signal through interpolation; generating the gradient information based on a differential value for each sample of the ECG data; and generating the waveform-specific interval information based on a numerical value of ECG signal waveforms included in the ECG data.

In an alternative embodiment, the generating of the waveform-specific interval information based on the numerical values of the ECG signal waveforms included in the ECG data may include extracting a feature value of each of the ECG signal waveforms included in the ECG data; deriving a numerical value corresponding to the feature value of each of the ECG signal waveforms, and normalizing each of the ECG signal waveforms based on the derived numerical value; and generating the waveform-specific interval information by combining numerical values of the respective normalized ECG signal waveforms.

In an alternative embodiment, the generating of the lead-specific integrated data may include generating the lead-specific integrated data by combining at least two of the ECG data, the gradient information, and the waveform-specific interval information.

In an alternative embodiment, the generating of the N-dimensional input data may include generating a matrix form of ND input data representing spatial information and time-series information of the ECG signal by arranging the lead-specific integrated data on a plane.

In an alternative embodiment, the method may further include predicting a chronic disease of a subject corresponding to the ECG signal based on the N-dimensional input data by using a pre-trained machine learning model.

In an alternative embodiment, the machine learning model may include an encoder extracting the feature by receiving the N-dimensional input data, and a decoder generating information on different types of chronic diseases based on the extracted feature.

In an alternative embodiment, the machine learning model may include an encoder extracting the feature by receiving the N-dimensional input data, and a decoder generating information on one chronic disease based on the extracted feature. In this case, when there are two or more decoders, the two or more decoders may generate information on different types of chronic diseases respectively.

In an alternative embodiment, the machine learning model may be trained based on N-dimensional training data including the spatial information and the time-series information of the ECG signal.

In an alternative embodiment, the method may further include generating a user interface based on information on the chronic disease predicted through the machine learning model.

In order to realize the object, disclosed is a computer program stored in a computer readable storage medium. The computer program performs operations for predicting a chronic disease based on an ECG signal when executed by one or more processors, and the operations may include: an operation of generating gradient information and waveform-specific interval information of an ECG signal from ECG data; an operation of generating lead-specific integrated data based on at least of the ECG data, the gradient information, or the waveform-specific interval information; and an operation of generating N-dimensional input data based on the lead-specific integrated data.

In order to realize the object, disclosed is a computing device for predicting a chronic disease based on an ECG signal. The device may include: a processor including at least one core; a memory including program codes executable in the processor; and a network unit for receiving an ECG signal, and the processor may generate gradient information and waveform-specific interval information of an ECG signal from ECG data; generate lead-specific integrated data based on at least one of ECG data, the gradient information or the waveform-specific interval information; and generate N-dimensional input data based on the lead-specific integrated data.

In order to realize the object, disclosed is a user terminal providing a user interface according to an embodiment of the present disclosure. The user terminal may include: a processor including at least one core; a memory; a network unit receiving a user interface based on analysis information of an ECG signal from a computing device; and an output unit providing the user interface. In this case, the analysis information of the ECG signal may include information on a chronic disease predicted through a pre-trained machine learning model, based on N-dimensional input data generated from the ECG signal.

Technical Benefits

The present disclosure can provide a method for predicting a chronic disease based on machine learning, which is configured to interpreting a mutual importance between leads for ECG measurement without losing unique information of an ECG signal.

### DESCRIPTION OF THE SEVERAL VIEWS OF THE DRAWINGS

FIG. 1 is a block diagram of a computing device for predicting a chronic disease based on an ECG signal according to an embodiment of the present disclosure.

FIG. 2 is a schematic view illustrating a network function according to an embodiment of the present disclosure.

FIG. 3 is a flowchart illustrating a preprocessing process for the ECG signal according to an embodiment of the present disclosure.

FIG. 4 is a conceptual diagram illustrating a process of generating integrated data for each lead according to an embodiment of the present disclosure.

FIG. 5 is a flowchart of a method for predicting a chronic disease based on an ECG signal according to an embodiment of the present disclosure.

FIG. 6 is a block diagram illustrating a structure of a machine learning model according to an embodiment of the present disclosure.

FIG. 7 is a block diagram illustrating a structure of a machine learning model according to an alternative embodiment of the present disclosure.

FIG. 8 is a schematic diagram of a computing environment according to an embodiment of the present disclosure.

### DETAILED DESCRIPTION

Various embodiments will now be described with reference to drawings. In the present specification, various descriptions are presented to provide appreciation of the present disclosure. However, it is apparent that the embodiments can be executed without the specific description.

“Component,” “module,” “system,” and the like which are terms used in the specification refer to a computer-related entity, hardware, firmware, software, and a combination of the software and the hardware, or execution of the software. For example, the component may be a processing process executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

Moreover, the term “or” is intended to mean not exclusive “or” but inclusive “or.” That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

Further, it should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

In addition, the term “at least one of A or B” should be interpreted to mean “a case including only A,” “a case including only B,” and “a case in which A and B are combined.”

Those skilled in the art need to recognize that various illustrative logical blocks, configurations, modules, circuits, means, logic, and algorithm steps described in connection with the embodiments disclosed herein may be additionally implemented as electronic hardware, computer software, or combinations of both sides. To clearly illustrate the interchangeability of hardware and software, various illustrative components, blocks, constitutions, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented embodiments is provided so that those skilled in the art of the present disclosure use or implement the present disclosure. Various modifications to the embodiments will be apparent to those skilled in the art. Generic principles defined herein may be applied to other embodiments without departing from the scope of the present disclosure. Therefore, the present disclosure is not limited to the embodiments presented herein. The present disclosure should be analyzed within the widest range which is coherent with the principles and new features presented herein.

In the present disclosure, a network function and an artificial neural network and a neural network may be interchangeably used.

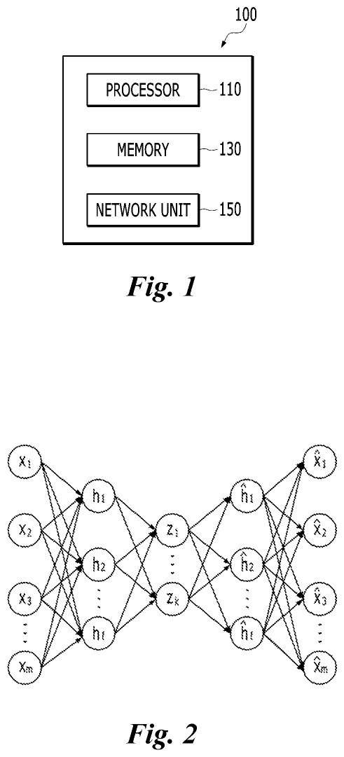

FIG. 1 is a block diagram of a computing device for predicting a chronic disease based on an ECG signal according to an embodiment of the present disclosure.

A configuration of the computing device 100 illustrated in FIG. 1 is only an example simplified and illustrated. In an exemplary embodiment of the present disclosure, the computing device 100 may include other components for performing a computing environment of the computing device 100, and only some of the disclosed components may constitute the computing device 100.

The computing device 100 may include a processor 110, a memory 130, and a network unit 150.

The processor 110 may be constituted by one or more cores, and include processors for data analysis and deep learning, such as a central processing unit (CPU), a general purpose graphics processing unit (GPGPU), a tensor processing unit (TPU), etc., of the computing device. The processor 110 may read a computer program stored in the memory 130 and process data for machine learning according to an exemplary embodiment of the present disclosure. According to an exemplary embodiment of the present disclosure, the processor 110 may perform an operation for learning the neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, the GPGPU, and the TPU of the processor 110 may process learning of the network function. For example, the CPU and the GPGPU may process the learning of the network function and data classification using the network function jointly. In addition, in an exemplary embodiment of the present disclosure, the learning of the network function and the data classification using the network function may be processed by using processors of a plurality of computing devices together. In addition, the computer program performed by the computing device according to an exemplary embodiment of the present disclosure may be a CPU, GPGPU, or TPU executable program.

According to an embodiment of the present disclosure, the processor 110 may perform preprocessing of converting an ECG signal into an input form of a machine learning model for predicting a chronic disease. The processor 110 may generate N-dimensional (N is a natural number) input data used for predicting the chronic disease based on a one-dimensional ECG signal measured through at least two or more leads. For example, the processor 110 may perform a sampling task of digitizing ECG signals that are analog measured with 12 leads. The processor 110 may extract unique information of ECG signals from ECG data generated through the sampling task, respectively. The processor 110 may generate 2D input data based on at least one of unique information of the ECG data and the ECG signals. The processor 110 may combine two or more of the unique information of the ECG data and the ECG signals for each lead, and generate the 2D input data based on a combination result. The 2D input data generated as described above may include both time information of the ECG signal and lead information of the ECG signal. Accordingly, the processor 110 may generate data interpretable by the machine learning model without losing time series information and spatial information of the ECG signal.

The processor 110 may train a machine learning model for predicting the chronic disease based on N-dimensional input data generated through preprocessing. For example, the processor 110 may input 2D input data to a model to train the model to predict whether a subject corresponding to the ECG signal suffers from a specific chronic disease. For example, the processor 110 may input the 2D input data to the model to train the model to estimate a quantitative value of a specific chronic disease suffered by the subject of the ECG signal. That is, according to a purpose of utilizing a prediction result of the chronic disease, the processor 110 may train the machine learning model variously based on the 2-dimensional input data generated by the preprocessing of the 1-dimensional ECG signal.

The processor 110 may predict the chronic disease based on the N-dimensional input data generated through the preprocessing for the ECG signal by using a pre-trained machine learning model. At this time, since the N-dimensional input data includes the lead information along with the time information of the ECG signal without losing both information, the machine learning model may output the unique information of the ECG signal and a chronic disease prediction result to interpret a mutual importance between leads. Accordingly, the processor 110 has an advantage in that there is no need to separately analyze the mutual importance of each lead or to retrain a machine learning model to determine information for each lead. In addition, the processor 110 has an advantage in that it is not necessary to separately process the correlation for each lead through a visualization task such as a heatmap so that a domain expert is configured to interpreting the correlation.

The processor 110 may generate a user interface based on the prediction result of the chronic disease generated through the machine learning model. At this time, the prediction result of the chronic disease output by the machine learning model is a probability of occurrence of various chronic diseases, including cardiovascular disease, brain disease, and lung disease, existence or nonexistence based on the probability of occurrence of each of the various chronic diseases, and quantitative values of chronic diseases from which the subject is currently suffering, etc. Depending on a purpose of utilizing the computing device 100 according to an embodiment of the present disclosure, an entire output result of the above example may be partitioned into regions and included in the user interface, or only a part of the output result may be included in the user interface.

According to an exemplary embodiment of the present disclosure, the memory 130 may store any type of information generated or determined by the processor 110 and any type of information received by the network unit 150.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

The network unit 150 according to the exemplary embodiment of the present disclosure may use a predetermined form of a publicly known wired/wireless communication system.

The network unit 150 may receive an ECG signal from a signal measurement system. In this case, the signal measurement system may be understood as a system including all devices configured to measuring, storing, and processing the ECG signal. For example, the signal measurement system may include a portable ECG measuring device including a lead contactable to a body of a subject, a database server that may be linked with the portable ECG measuring device, and the like. The network unit 150 may receive an ECG signal measured through leads having various two or more combinations through communication with the portable ECG measurement device. The network unit 150 may receive an ECG signal previously measured by the portable ECG measuring device and stored in the database server through communication with the database server.

In addition, the network unit 150 may transmit and receive information processed by the processor 110, a user interface, and the like through communication with other terminals. For example, the network unit 150 may provide the user interface generated by the processor 110 to a client (e.g., a user terminal). In addition, the network unit 150 may receive an external input of a user applied to a client and transfer the external input to the processor 110. In this case, the processor 110 may process operations such as outputting, correcting, changing, adding, and the like of information provided through the user interface based on the external input of the user received from the network unit 150.

Meanwhile, according to an embodiment of the present disclosure, the computing device 100 may include a server as a computing system that transmits and receives information through communication with the client. In this case, the client may be any type of terminal which may access the server. For example, the computing device 100 as the server may receive the ECG signal from the signal measurement system, predict a chronic disease, and provide a user interface including a predicted result to the user terminal. At this time, the user terminal may output the user interface received from the computing device 100 as the server, and receive or process information through interaction with the user.

The user terminal may display the user interface provided to provide analysis information on the chronic disease transmitted from the computing device 100 as the server. Although not separately illustrated, the user terminal may include a network unit receiving the user interface from the computing device 100, a processor including at least one core, a memory, an output unit providing the user interface, and input unit receiving the external input applied from the user.

In an additional embodiment, the computing device 100 may also include any type of terminal that receives data resources generated by an arbitrary server and performs additional information processing.

FIG. 2 is a schematic diagram illustrating a network function according to the embodiment of the present disclosure.

Throughout the present specification, the meanings of a calculation model, a nerve network, the network function, and the neural network may be interchangeably used. The neural network may be formed of a set of interconnected calculation units which are generally referred to as “nodes.” The “nodes” may also be called “neurons.” The neural network consists of one or more nodes. The nodes (or neurons) configuring the neural network may be interconnected by one or more links.

In the neural network, one or more nodes connected through the links may relatively form a relationship of an input node and an output node. The concept of the input node is relative to the concept of the output node, and a predetermined node having an output node relationship with respect to one node may have an input node relationship in a relationship with another node, and a reverse relationship is also available. As described above, the relationship between the input node and the output node may be generated based on the link. One or more output nodes may be connected to one input node through a link, and a reverse case may also be valid.

In the relationship between an input node and an output node connected through one link, a value of the output node data may be determined based on data input to the input node. Herein, a link connecting the input node and the output node may have a weight. The weight is variable, and in order for the neural network to perform a desired function, the weight may be varied by a user or an algorithm. For example, when one or more input nodes are connected to one output node by links, respectively, a value of the output node may be determined based on values input to the input nodes connected to the output node and weights set in the link corresponding to each of the input nodes.

As described above, in the neural network, one or more nodes are connected with each other through one or more links to form a relationship of an input node and an output node in the neural network. A characteristic of the neural network may be determined according to the number of nodes and links in the neural network, a correlation between the nodes and the links, and a value of the weight assigned to each of the links. For example, when there are two neural networks in which the numbers of nodes and links are the same and the weight values between the links are different, the two neural networks may be recognized to be different from each other.

The neural network may consist of a set of one or more nodes. A subset of the nodes configuring the neural network may form a layer. Some of the nodes configuring the neural network may form one layer on the basis of distances from an initial input node. For example, a set of nodes having a distance of n from an initial input node may form n layers. The distance from the initial input node may be defined by the minimum number of links, which need to be passed to reach a corresponding node from the initial input node. However, the definition of the layer is arbitrary for the description, and a degree of the layer in the neural network may be defined by a different method from the foregoing method. For example, the layers of the nodes may be defined by a distance from a final output node.

The initial input node may mean one or more nodes to which data is directly input without passing through a link in a relationship with other nodes among the nodes in the neural network. Otherwise, the initial input node may mean nodes which do not have other input nodes connected through the links in a relationship between the nodes based on the link in the neural network. Similarly, the final output node may mean one or more nodes that do not have an output node in a relationship with other nodes among the nodes in the neural network. Further, the hidden node may mean nodes configuring the neural network, not the initial input node and the final output node.

In the neural network according to the embodiment of the present disclosure, the number of nodes of the input layer may be the same as the number of nodes of the output layer, and the neural network may be in the form that the number of nodes decreases and then increases again from the input layer to the hidden layer. Further, in the neural network according to another embodiment of the present disclosure, the number of nodes of the input layer may be smaller than the number of nodes of the output layer, and the neural network may be in the form that the number of nodes decreases from the input layer to the hidden layer. Further, in the neural network according to another embodiment of the present disclosure, the number of nodes of the input layer may be larger than the number of nodes of the output layer, and the neural network may be in the form that the number of nodes increases from the input layer to the hidden layer. The neural network according to another embodiment of the present disclosure may be the neural network in the form in which the foregoing neural networks are combined.

A deep neural network (DNN) may mean the neural network including a plurality of hidden layers, in addition to an input layer and an output layer. When the DNN is used, it is possible to recognize a latent structure of data. That is, it is possible to recognize latent structures of photos, texts, videos, voice, and music (for example, what objects are in the photos, what the content and emotions of the texts are, and what the content and emotions of the voice are). The DNN may include a convolutional neural network (CNN), a recurrent neural network (RNN), an auto encoder, Generative Adversarial Networks (GAN), a Long Short-Term Memory (LSTM), a transformer, a restricted Boltzmann machine (RBM), a deep belief network (DBN), a Q network, a U network, a Siamese network, a Generative Adversarial Network (GAN), and the like. The foregoing description of the deep neural network is merely illustrative, and the present disclosure is not limited thereto.

In the embodiment of the present disclosure, the network function may include an auto encoder. The auto encoder may be one type of artificial neural network for outputting output data similar to input data. The auto encoder may include at least one hidden layer, and the odd-numbered hidden layers may be disposed between the input/output layers. The number of nodes of each layer may decrease from the number of nodes of the input layer to an intermediate layer called a bottleneck layer (encoding), and then be expanded symmetrically with the decrease from the bottleneck layer to the output layer (symmetric with the input layer). The auto encoder may perform a nonlinear dimension reduction. The number of input layers and the number of output layers may correspond to the dimensions after preprocessing of the input data. In the auto encoder structure, the number of nodes of the hidden layer included in the encoder decreases as a distance from the input layer increases. When the number of nodes of the bottleneck layer (the layer having the smallest number of nodes located between the encoder and the decoder) is too small, the sufficient amount of information may not be transmitted, so that the number of nodes of the bottleneck layer may be maintained in a specific number or more (for example, a half or more of the number of nodes of the input layer and the like).

The neural network may be trained by at least one scheme of supervised learning, unsupervised learning, semi-supervised learning, and reinforcement learning. The training of the neural network may be a process of applying knowledge for the neural network to perform a specific operation to the neural network.

The neural network may be trained in a direction of minimizing an error of an output. In the training of the neural network, training data is repeatedly input to the neural network and an error of an output of the neural network for the training data and a target is calculated, and the error of the neural network is back-propagated in a direction from an output layer to an input layer of the neural network in order to decrease the error, and a weight of each node of the neural network is updated. In the case of the supervised learning, training data labelled with a correct answer (that is, labelled training data) is used, in each training data, and in the case of the unsupervised learning, a correct answer may not be labelled to each training data. That is, for example, the training data in the supervised learning for data classification may be data, in which category is labelled to each of the training data. The labelled training data is input to the neural network and the output (category) of the neural network is compared with the label of the training data to calculate an error. For another example, in the case of the unsupervised learning related to the data classification, training data that is the input is compared with an output of the neural network, so that an error may be calculated. The calculated error is back-propagated in a reverse direction (that is, the direction from the output layer to the input layer) in the neural network, and a connection weight of each of the nodes of the layers of the neural network may be updated according to the backpropagation. A change amount of the updated connection weight of each node may be determined according to a learning rate. The calculation of the neural network for the input data and the backpropagation of the error may configure a learning epoch. The learning rate is differently applicable according to the number of times of repetition of the learning epoch of the neural network. For example, at the initial stage of the learning of the neural network, a high learning rate is used to make the neural network rapidly secure performance of a predetermined level and improve efficiency, and at the latter stage of the learning, a low learning rate is used to improve accuracy.

In the training of the neural network, the training data may be generally a subset of actual data (that is, data to be processed by using the trained neural network), and thus an error for the training data is decreased, but there may exist a learning epoch, in which an error for the actual data is increased. Overfitting is a phenomenon, in which the neural network excessively learns training data, so that an error for actual data is increased. For example, a phenomenon, in which the neural network learning a cat while seeing a yellow cat cannot recognize cats, other than a yellow cat, as cats, is a sort of overfitting. Overfitting may act as a reason of increasing an error of a machine learning algorithm. In order to prevent overfitting, various optimizing methods may be used. In order to prevent overfitting, a method of increasing training data, a regularization method, a dropout method of inactivating a part of nodes of the network during the training process, a method using a bath normalization layer, and the like may be applied.

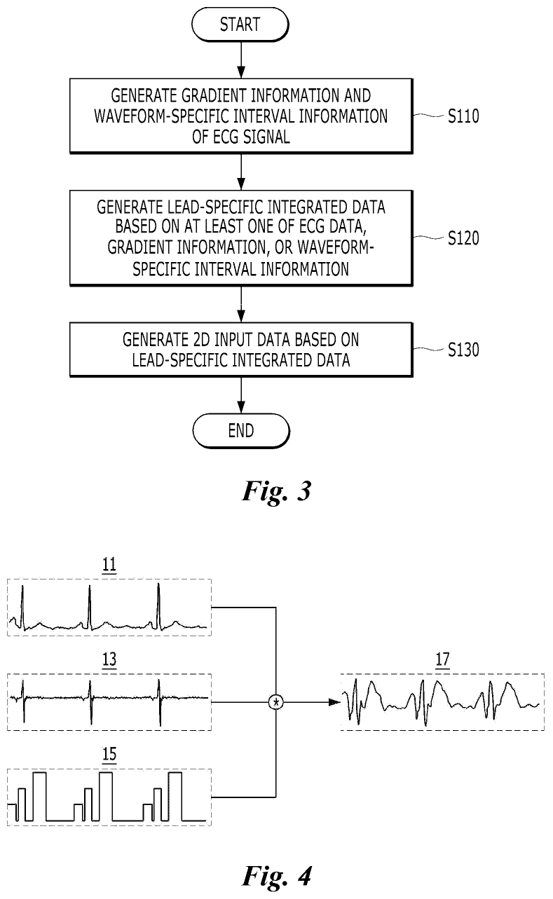

FIG. 3 is a flowchart illustrating a preprocessing process for the ECG signal according to an embodiment of the present disclosure. In addition, FIG. 4 is a conceptual diagram illustrating a process of generating integrated data for each lead according to an embodiment of the present disclosure. Hereinafter, a process of preprocessing and predicting a chronic disease according to an embodiment of the present disclosure will be described based on two-dimensional input data, which is one of N-dimensional input data.

Referring to FIG. 3, in step S110, a computing device 100 according to an embodiment of the present disclosure may generate ECG data by digitally sampling an ECG signal. The computing device 100 may generate the ECG data, which is a digital signal, from the ECG signal, which is an analog signal, through interpolation. The interpolation may be understood as a task of reconstructing missing values between samples made by capturing analog amplitudes at specific time intervals. In this case, how many time intervals to capture signals and generate samples may be determined based on a predetermined sample rate. For example, the computing device 100 may process the ECG signal by performing the interpolation based on the predetermined sample rate according to a pre-trained machine learning model. The computing device 100 may process the ECG signal sampled through the interpolation into a length corresponding to an input length of the model. The computing device 100 may generate the ECG data from the ECG signal through the interpolation and length processing.

In step S110, the computing device 100 may generate gradient information of the ECG signal from the ECG data. The computing device 100 may generate the gradient information of the ECG signal based on a differential value for each sample of the ECG data. In this case, the gradient information may be understood as unique information of the ECG signal representing a direction of a peak value of the ECG signal. For example, assuming that there are N (N is a natural number) samples of ECG data generated through the above process, the computing device 100 may calculate the differential value based on the N samples to calculate N−1 gradient information. The computing device 100 may calculate insufficient final gradient information for the number of samples using various padding methods such as constant padding and symmetric padding. That is, the computing device 100 may generate the same number of gradient information as samples of the ECG data by making gradient information calculated as N−1 pieces into N pieces through padding.

The computing device 100 may generate interval information for each waveform of the ECG signal from the ECG data. The computing device 100 may generate the interval information for each waveform based on numerical values of ECG signal waveforms included in the ECG data. For example, the waveform of the ECG signal reflecting an electrical activation phase of the heart may be basically divided into a P wave, a QRS-complex, a T wave, and other waveforms. The computing device 100 may normalize different numerical values for the P wave, the QRS-complex, the T wave, and other corresponding waveforms. In this case, the normalized numerical values may also include peak values of P, Q, R, S, and T waves. The computing device 100 may generate the interval information for each waveform of the ECG signal by configuring the normalized numerical values for each waveform based on the time series information of the ECG data to have a length corresponding to the ECG data.

Specifically, the computing device 100 may extract feature values of the respective ECG signal waveforms included in the ECG data. For example, the computing device 100 may extract feature values of waveforms constituting the ECG signal, such as the P wave, the QRS-complex, and the T wave, by using a pre-trained deep learning model. At this time, the feature value may include unique information of the respective ECG signal waveforms including the P wave, the QRS-complex, and the T-wave, such as an onset point time of the P wave, an offset point time of the P wave, and an onset point time of the QRS-complex. The computing device 100 may also extract the feature value of each signal waveform based on a specific rule.

The computing device 100 may derive a numerical value of each signal waveform corresponding to the feature value of each signal waveform. The computing device 100 may process the waveforms constituting the ECG signal into a form corresponding to the ECG signal based on the numerical value of each signal waveform. The computing device 100 may generate the interval information for each waveform by combining the ECG signal waveforms processed in the form corresponding to the ECG signal. For example, in the computing device 100, the numerical value corresponding to the feature value of the P wave may be derived as 1, the numerical value corresponding to the feature value of the QRS-complex may be derived as 2, and the numerical value corresponding to the feature value of the T wave may be derived as 3, and numerical values corresponding to feature values of the remaining waveforms may be derived as 0. The computing device 100 may normalize the P wave, the QRS-complex, the T wave, and the remaining waveforms based on the numerical value of each waveform. The computing device 100 may generate one waveform-specific interval information by combining the normalized P wave, QRS-complex, T wave, and remaining waveforms. Meanwhile, since specific numerical values such as 1, 2, 3, and 0 are only examples, the numerical values may be changed to other values within a range that may be understood by those skilled in the art.

In step S120, the computing device 100 may generate integrated data for each lead based on at least one of the ECG data, gradient information, and interval information for each waveform generated through step S110. The computing device 100 may generate the integrated data for each read by combining two or more of the three pieces of data regardless of order. In this case, the number and type of data to be combined to generate the integrated data for each lead may vary depending on the purpose of utilizing the chronic disease prediction result. For example, as illustrated in FIG. 4, the computing device 100 may perform a convolution operation by combining ECG data 11, gradient information 13, and waveform-specific interval information 15. In other words, the computing device 100 combines the ECG data 11, the gradient information 13, and the waveform-specific interval information 15 by performing the convolution operation to generate specific-lead integrated data 17. However, an operation for combining data may include all operations that may be applied to combine data, such as a multiplication operation, a sum operation, an average operation, and the like, in addition to the convolution operation described above. The lead-specific integrated data generated through this process helps the machine learning model perform data interpretation to predict the chronic disease with information and criteria similar to that of humans, which are domain experts.

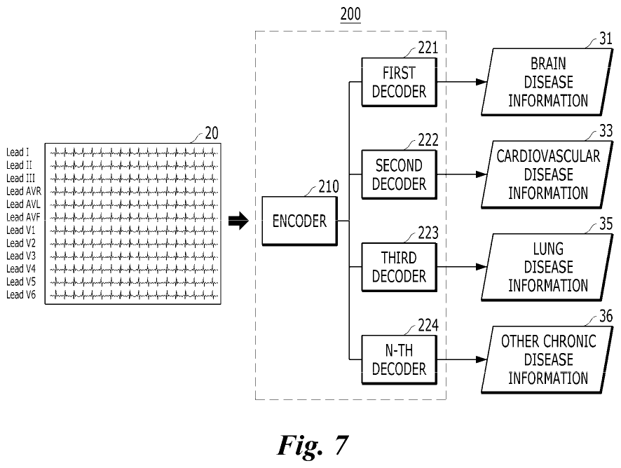

In step S130, the computing device 100 may generate two-dimensional input data used for an input of a machine learning model for predicting the chronic disease based on the lead-specific integrated data generated in step S120. In this case, the 2D input data may be in the form of a matrix representing time-series information and spatial information of the ECG signal. For example, the computing device 100 arranges the lead-specific integrated data on a plane based on the same time to generate input data in the form of a two-dimensional matrix in which an X axis represents the time series information and a Y axis represents lead information. For example, if the number of leads used to measure the ECG signal is K (K is a natural number), the computing device 100 aligns the integrated data for each K lead at the same time based on a time interval T on a plane to generate input data having a K×T two-dimensional matrix form. An example of the 2D input data may be confirmed through an image 20 illustrated in FIG. 6 or 7 to be described later. The two-dimensional input data generated through this process enables integrated processing of the spatial information of the ECG signal to help lead-specific correlation required for diagnosing a specific chronic disease be effectively reflected in the inference process of the machine learning model.

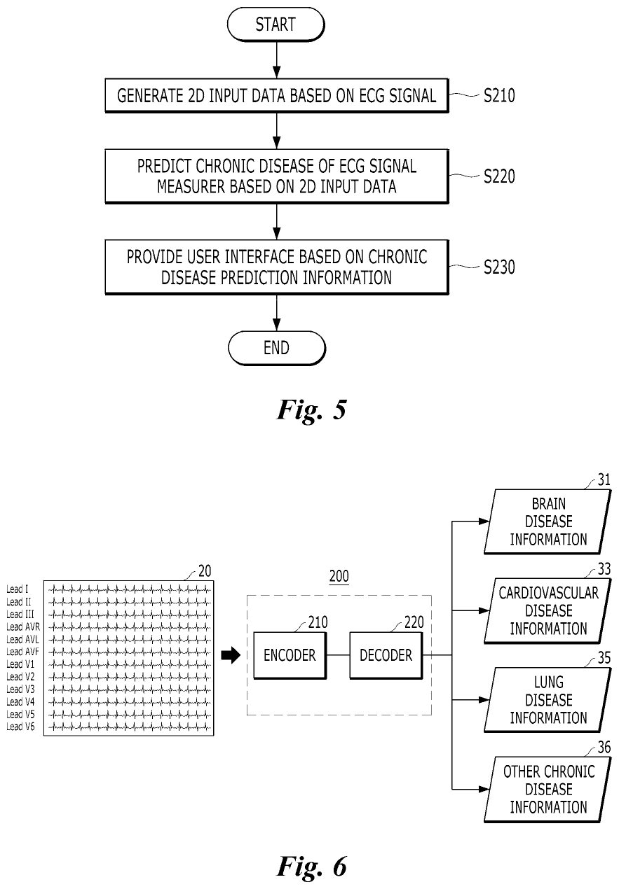

FIG. 5 is a flowchart of a method for predicting a chronic disease based on an ECG signal according to an embodiment of the present disclosure.

Referring to FIG. 5, step S210 of generating two-dimensional input data based on an ECG signal according to an embodiment of the present disclosure may be understood to correspond to the previous steps of FIG. 3 described above. Therefore, an additional description of step S210 will be omitted.

In step S220, the computing device 100 according to an embodiment of the present disclosure may predict a chronic disease of a subject who measures an ECG signal based on the two-dimensional input data generated in step S210 by using a pre-trained machine learning model. For example, the computing device 100 may input 2D input data in which time-series information and spatial information of the ECG signal are arranged in a matrix form into the machine learning model. The machine learning model may infer the presence or absence of various types of chronic diseases and quantitative values based on features present in the two-dimensional input data. In this case, the machine learning model may also predict the chronic disease by receiving biological information related to the subject corresponding to the ECG signal, environmental information, and the like jointly. Chronic diseases which may be predicted by the machine learning model may include cardiovascular diseases such as arrhythmia, heart failure, and myocardial infarction, brain diseases such as cerebral hemorrhage, cerebral infarction, and stroke, lung diseases such as pulmonary embolism, and other chronic diseases such as diabetes and hypertension. Accordingly, the computing device 100 may derive probability information, linear numerical information, and the like for all or some of the chronic diseases predictable by the machine learning model as analysis information of the ECG signal according to the purpose of utilization.

In step S230, the computing device 100 may generate a user interface based on analysis information of the ECG signal, which is the prediction result of the chronic disease generated in step S220. The computing device 100 may generate the user interface based on all or part of ECG signal analysis information including probability information indicating the presence or absence of a specific chronic disease, linear numerical information indicating the severity of a specific chronic disease, and the like. For example, the computing device 100 may generate a user interface including a first region representing the probability information about the presence or absence of the cardiovascular disease and a second region representing the linear numerical information about the severity of the cardiovascular disease. The computing device 100 may provide a user interface for outputting a prediction result of the chronic disease to a user terminal through communication with the user terminal.

Each of FIGS. 6 and 7 is a block diagram illustrating a structure of a machine learning model according to an embodiment of the present disclosure.

Referring to FIG. 6, a machine learning model 200 according to an embodiment of the present disclosure may include an encoder 210 extracting features by receiving two-dimensional input data 20 and a decoder 220 generating information 31, 33, 35, and 36 on different types of chronic diseases based on the feature extracted by the encoder. Since the machine learning model 200 receives the two-dimensional input data 20 including the spatial information about the lead of the ECG signal, it is possible to extract features of the input data through one encoder 210 unlike conventional schemes. Therefore, compared to a conventional one-dimensional model or a two-dimensional model based on frequency conversion, the model may be made lighter and a data processing speed of the model may be significantly improved. In addition, since the machine learning model 200 is configured to performing integrated processing of the spatial information through the use of the two-dimensional input data 20, the accuracy of prediction and judgment of a specific chronic disease may be significantly improved compared to conventional models.

The machine learning model 200 may generate information 31, 33, 35, and 36 on different types of chronic diseases based on a feature of the 2D input data 20 extracted through the encoder 210 through one decoder 220. For example, the decoder 220 may output at least one of brain disease information 31, cardiovascular disease information 33, lung disease information 35, and other chronic disease information 36 such as diabetes based on the feature of the two-dimensional input data 20. In this case, the information on the specific chronic disease may include a result of predicting whether the specific chronic disease exists, a result of judging a quantitative value related to the specific chronic disease, and the like. The decoder 220 may also selectively output the brain disease information 31, the cardiovascular disease information 33, the lung disease information 35, and other chronic disease information 36 though the control of the computing device 100 for the machine learning model 200.

Referring to FIG. 7, the machine learning model 200 according to an alternative embodiment of the present disclosure may include a plurality of decoders 221, 222, 223, and 224 that generate information 31, 33, 35, and 36 on different types of chronic diseases based on the features extracted by the encoder. Unlike FIG. 6, the machine learning model 200 may include a plurality of decoders 221, 222, 223, and 224 individually corresponding to different types of chronic diseases. For example, the machine learning model 200 may include a first decoder 221 generating the brain disease information 31, a second decoder 222 generating the cardiovascular disease information 33, a third decoder 223 generating the lung disease information 35, and an N-th decoder 224 generating other chronic disease information 36. At this time, when other chronic diseases are further subdivided, the N-th decoder 224 may also be subdivided into several decoders. The machine learning model 200 may selectively generate information on various chronic diseases by independently operating the plurality of decoders 221, 222, 223, and 224.

The types of chronic diseases described above are only some examples, and various types of examples may be applied within the range that those skilled in the art can understand.

In the meantime, according to an embodiment of the present disclosure, a computer readable medium storing a data structure is disclosed.

The data structure may refer to organization, management, and storage of data that enable efficient access and modification of data. The data structure may refer to organization of data for solving a specific problem (for example, data search, data storage, and data modification in the shortest time). The data structure may also be defined with a physical or logical relationship between the data elements designed to support a specific data processing function. A logical relationship between data elements may include a connection relationship between user defined data elements. A physical relationship between data elements may include an actual relationship between the data elements physically stored in a computer readable storage medium (for example, a permanent storage device). In particular, the data structure may include a set of data, a relationship between data, and a function or a command applicable to data. Through the effectively designed data structure, the computing device may perform a calculation while minimally using resources of the computing device. In particular, the computing device may improve efficiency of calculation, reading, insertion, deletion, comparison, exchange, and search through the effectively designed data structure.

The data structure may be divided into a linear data structure and a non-linear data structure according to the form of the data structure. The linear data structure may be the structure in which only one data is connected after one data. The linear data structure may include a list, a stack, a queue, and a deque. The list may mean a series of dataset in which order exists internally. The list may include a linked list. The linked list may have a data structure in which data is connected in a method in which each data has a pointer and is linked in a single line. In the linked list, the pointer may include information about the connection with the next or previous data. The linked list may be expressed as a single linked list, a double linked list, and a circular linked list according to the form. The stack may have a data listing structure with limited access to data. The stack may have a linear data structure that may process (for example, insert or delete) data only at one end of the data structure. The data stored in the stack may have a data structure (Last In First Out, LIFO) in which the later the data enters, the sooner the data comes out. The queue is a data listing structure with limited access to data, and may have a data structure (First In First Out, FIFO) in which the later the data is stored, the later the data comes out, unlike the stack. The deque may have a data structure that may process data at both ends of the data structure.

The non-linear data structure may be the structure in which the plurality of data is connected after one data. The non-linear data structure may include a graph data structure. The graph data structure may be defined with a vertex and an edge, and the edge may include a line connecting two different vertexes. The graph data structure may include a tree data structure. The tree data structure may be the data structure in which a path connecting two different vertexes among the plurality of vertexes included in the tree is one. That is, the tree data structure may be the data structure in which a loop is not formed in the graph data structure.

Throughout the present specification, a calculation model, a nerve network, the network function, and the neural network may be used with the same meaning. Hereinafter, the terms of the calculation model, the nerve network, the network function, and the neural network are unified and described with a neural network. The data structure may include a neural network. Further, the data structure including the neural network may be stored in a computer readable medium. The data structure including the neural network may also include preprocessed data for processing by the neural network, data input to the neural network, a weight of the neural network, a hyper-parameter of the neural network, data obtained from the neural network, an active function associated with each node or layer of the neural network, and a loss function for training of the neural network. The data structure including the neural network may include predetermined configuration elements among the disclosed configurations. That is, the data structure including the neural network may include the entirety or a predetermined combination of pre-processed data for processing by neural network, data input to the neural network, a weight of the neural network, a hyper parameter of the neural network, data obtained from the neural network, an active function associated with each node or layer of the neural network, and a loss function for training the neural network. In addition to the foregoing configurations, the data structure including the neural network may include predetermined other information determining a characteristic of the neural network. Further, the data structure may include all type of data used or generated in a computation process of the neural network, and is not limited to the foregoing matter. The computer readable medium may include a computer readable recording medium and/or a computer readable transmission medium. The neural network may be formed of a set of interconnected calculation units which are generally referred to as “nodes.” The “nodes” may also be called “neurons.” The neural network consists of one or more nodes.

The data structure may include data input to the neural network. The data structure including the data input to the neural network may be stored in the computer readable medium. The data input to the neural network may include training data input in the training process of the neural network and/or input data input to the training completed neural network. The data input to the neural network may include data that has undergone pre-processing and/or data to be pre-processed. The pre-processing may include a data processing process for inputting data to the neural network. Accordingly, the data structure may include data to be pre-processed and data generated by the pre-processing. The foregoing data structure is merely an example, and the present disclosure is not limited thereto.

The data structure may include a weight of the neural network (in the present specification, weights and parameters may be used with the same meaning), Further, the data structure including the weight of the neural network may be stored in the computer readable medium. The neural network may include a plurality of weights. The weight is variable, and in order for the neural network to perform a desired function, the weight may be varied by a user or an algorithm. For example, when one or more input nodes are connected to one output node by links, respectively, the output node may determine a data value output from the output node based on values input to the input nodes connected to the output node and the weight set in the link corresponding to each of the input nodes. The foregoing data structure is merely an example, and the present disclosure is not limited thereto.

For a non-limited example, the weight may include a weight varied in the neural network training process and/or the weight when the training of the neural network is completed. The weight varied in the neural network training process may include a weight at a time at which a training cycle starts and/or a weight varied during a training cycle. The weight when the training of the neural network is completed may include a weight of the neural network completing the training cycle. Accordingly, the data structure including the weight of the neural network may include the data structure including the weight varied in the neural network training process and/or the weight when the training of the neural network is completed. Accordingly, it is assumed that the weight and/or a combination of the respective weights are included in the data structure including the weight of the neural network. The foregoing data structure is merely an example, and the present disclosure is not limited thereto.

The data structure including the weight of the neural network may be stored in the computer readable storage medium (for example, a memory and a hard disk) after undergoing a serialization process. The serialization may be the process of storing the data structure in the same or different computing devices and converting the data structure into a form that may be reconstructed and used later. The computing device may serialize the data structure and transceive the data through a network. The serialized data structure including the weight of the neural network may be reconstructed in the same or different computing devices through deserialization. The data structure including the weight of the neural network is not limited to the serialization. Further, the data structure including the weight of the neural network may include a data structure (for example, in the non-linear data structure, B-Tree, Trie, m-way search tree, AVL tree, and Red-Black Tree) for improving efficiency of the calculation while minimally using the resources of the computing device. The foregoing matter is merely an example, and the present disclosure is not limited thereto.

The data structure may include a hyper-parameter of the neural network. The data structure including the hyper-parameter of the neural network may be stored in the computer readable medium. The hyper-parameter may be a variable varied by a user. The hyper-parameter may include, for example, a learning rate, a cost function, the number of times of repetition of the training cycle, weight initialization (for example, setting of a range of a weight value to be weight-initialized), and the number of hidden units (for example, the number of hidden layers and the number of nodes of the hidden layer). The foregoing data structure is merely an example, and the present disclosure is not limited thereto.

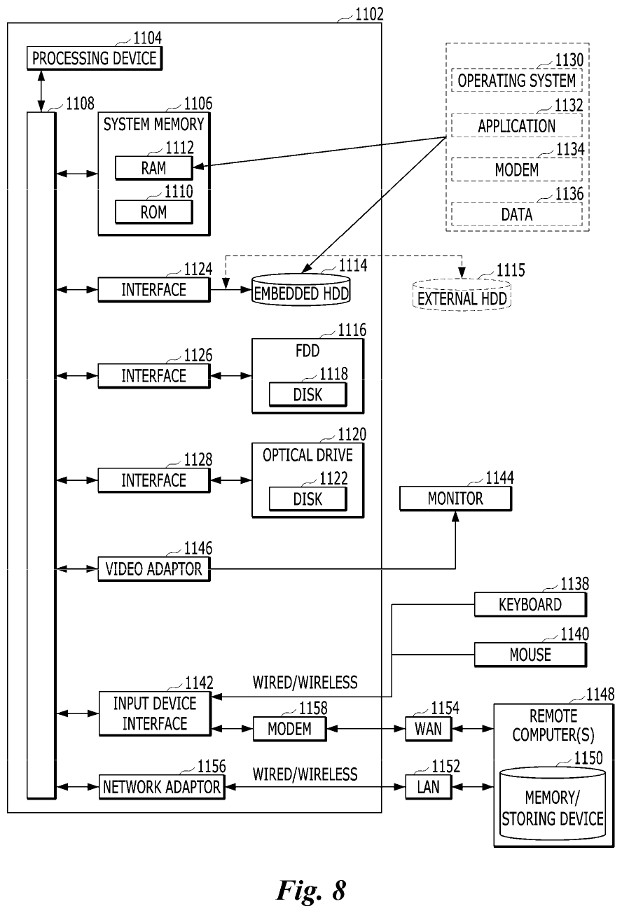

FIG. 8 is a simple and general schematic diagram illustrating an example of a computing environment in which the embodiments of the present disclosure are implementable.

The present disclosure has been described as being generally implementable by the computing device, but those skilled in the art will appreciate well that the present disclosure is combined with computer executable commands and/or other program modules executable in one or more computers and/or be implemented by a combination of hardware and software.

In general, a program module includes a routine, a program, a component, a data structure, and the like performing a specific task or implementing a specific abstract data form. Further, those skilled in the art will well appreciate that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium. The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

An illustrative environment 1100 including a computer 1102 and implementing several aspects of the present disclosure is illustrated, and the computer 1102 includes a processing device 1104, a system memory 1106, and a system bus 1108. The system bus 1108 connects system components including the system memory 1106 (not limited) to the processing device 1104. The processing device 1104 may be a predetermined processor among various commonly used processors. A dual processor and other multi-processor architectures may also be used as the processing device 1104.

The system bus 1108 may be a predetermined one among several types of bus structure, which may be additionally connectable to a local bus using a predetermined one among a memory bus, a peripheral device bus, and various common bus architectures. The system memory 1106 includes a ROM 1110, and a RAM 1112. A basic input/output system (BIOS) is stored in a non-volatile memory 1110, such as a ROM, an EPROM, and an EEPROM, and the BIOS includes a basic routing helping a transport of information among the constituent elements within the computer 1102 at a time, such as starting. The RAM 1112 may also include a high-rate RAM, such as a static RAM, for caching data.

The computer 1102 also includes an embedded hard disk drive (HDD) 1114 (for example, enhanced integrated drive electronics (EIDE) and serial advanced technology attachment (SATA))—the embedded HDD 1114 being configured for exterior mounted usage within a proper chassis (not illustrated)—a magnetic floppy disk drive (FDD) 1116 (for example, which is for reading data from a portable diskette 1118 or recording data in the portable diskette 1118), and an optical disk drive 1120 (for example, which is for reading a CD-ROM disk 1122, or reading data from other high-capacity optical media, such as a DVD, or recording data in the high-capacity optical media). A hard disk drive 1114, a magnetic disk drive 1116, and an optical disk drive 1120 may be connected to a system bus 1108 by a hard disk drive interface 1124, a magnetic disk drive interface 1126, and an optical drive interface 1128, respectively. An interface 1124 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1102, the drive and the medium correspond to the storage of random data in an appropriate digital form. In the description of the computer readable media, the HDD, the portable magnetic disk, and the portable optical media, such as a CD, or a DVD, are mentioned, but those skilled in the art will well appreciate that other types of computer readable media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1130, one or more application programs 1132, other program modules 1134, and program data 1136 may be stored in the drive and the RAM 1112. An entirety or a part of the operation system, the application, the module, and/or data may also be cached in the RAM 1112. It will be well appreciated that the present disclosure may be implemented by several commercially usable operation systems or a combination of operation systems.

A user may input a command and information to the computer 1102 through one or more wired/wireless input devices, for example, a keyboard 1138 and a pointing device, such as a mouse 1140. Other input devices (not illustrated) may be a microphone, an IR remote controller, a joystick, a game pad, a stylus pen, a touch screen, and the like. The foregoing and other input devices are frequently connected to the processing device 1104 through an input device interface 1142 connected to the system bus 1108, but may be connected by other interfaces, such as a parallel port, an IEEE 1394 serial port, a game port, a USB port, an IR interface, and other interfaces.

A monitor 1144 or other types of display devices are also connected to the system bus 1108 through an interface, such as a video adaptor 1146. In addition to the monitor 1144, the computer generally includes other peripheral output devices (not illustrated), such as a speaker and a printer.

The computer 1102 may be operated in a networked environment by using a logical connection to one or more remote computers, such as remote computer(s) 1148, through wired and/or wireless communication. The remote computer(s) 1148 may be a work station, a computing device computer, a router, a personal computer, a portable computer, a microprocessor-based entertainment device, a peer device, and other general network nodes, and generally includes some or an entirety of the constituent elements described for the computer 1102, but only a memory storage device 1150 is illustrated for simplicity. The illustrated logical connection includes a wired/wireless connection to a local area network (LAN) 1152 and/or a larger network, for example, a wide area network (WAN) 1154. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1102 is used in the LAN networking environment, the computer 1102 is connected to the local network 1152 through a wired and/or wireless communication network interface or an adaptor 1156. The adaptor 1156 may make wired or wireless communication to the LAN 1152 easy, and the LAN 1152 also includes a wireless access point installed therein for the communication with the wireless adaptor 1156. When the computer 1102 is used in the WAN networking environment, the computer 1102 may include a modem 1158, is connected to a communication computing device on a WAN 1154, or includes other means setting communication through the WAN 1154 via the Internet. The modem 1158, which may be an embedded or outer-mounted and wired or wireless device, is connected to the system bus 1108 through a serial port interface 1142. In the networked environment, the program modules described for the computer 1102 or some of the program modules may be stored in a remote memory/storage device 1150. The illustrated network connection is illustrative, and those skilled in the art will appreciate well that other means setting a communication link between the computers may be used.

The computer 1102 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

The Wi-Fi enables a connection to the Internet and the like even without a wire. The Wi-Fi is a wireless technology, such as a cellular phone, which enables the device, for example, the computer, to transmit and receive data indoors and outdoors, that is, in any place within a communication range of a base station. A Wi-Fi network uses a wireless technology, which is called IEEE 802.11 (a, b, g, etc.) for providing a safe, reliable, and high-rate wireless connection. The Wi-Fi may be used for connecting the computer to the computer, the Internet, and the wired network (IEEE 802.3 or Ethernet is used). The Wi-Fi network may be operated at, for example, a data rate of 11 Mbps (802.11a) or 54 Mbps (802.11b) in an unauthorized 2.4 and 5 GHz wireless band, or may be operated in a product including both bands (dual bands).

Those skilled in the art may appreciate that information and signals may be expressed by using predetermined various different technologies and techniques. For example, data, indications, commands, information, signals, bits, symbols, and chips referable in the foregoing description may be expressed with voltages, currents, electromagnetic waves, magnetic fields or particles, optical fields or particles, or a predetermined combination thereof.

Those skilled in the art will appreciate that the various illustrative logical blocks, modules, processors, means, circuits, and algorithm operations described in relationship to the embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various embodiments presented herein may be implemented by a method, a device, or a manufactured article using a standard programming and/or engineering technology. A term “manufactured article” includes a computer program, a carrier, or a medium accessible from a predetermined computer-readable storage device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. Further, various storage media presented herein include one or more devices and/or other machine-readable media for storing information.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

The description of the presented embodiments is provided so as for those skilled in the art to use or carry out the present disclosure. Various modifications of the embodiments may be apparent to those skilled in the art, and general principles defined herein may be applied to other embodiments without departing from the scope of the present disclosure. Accordingly, the present disclosure is not limited to the embodiments suggested herein, and shall be interpreted within the broadest meaning range consistent to the principles and new characteristics presented herein.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for predicting a chronic disease based on an ECG signal performed by a computing device including at least one processor, the method comprising:
generating lead-specific integrated data based on the ECG signal, and generating N-dimensional input data based on the lead-specific integrated data;
predicting the chronic disease through a pre-trained machine learning model based on the N-dimensional input data; and
generating prediction information on the chronic disease to be provided to a user.

2. The method of claim 1, wherein the lead-specific integrated data is generated based on at least one of gradient information of the ECG signal or waveform-specific interval information of the ECG signal.

3. The method of claim 2, wherein the generating of the N-dimensional input data includes:
generating at least one of the waveform-specific interval information of the ECG signal or the gradient information of the ECG signal;
generating the lead-specific integrated data based on at least one of the waveform-specific interval information of the ECG signal or the gradient information of the ECG signal; and
generating the N-dimensional input data based on the lead-specific integrated data.

4. The method of claim 3, wherein the generating of at least one of the waveform-specific interval information of the ECG signal or the gradient information of the ECG signal includes:
generating ECG data by sampling the ECG signal through interpolation;
generating the gradient information based on a differential value for each sample of the ECG data; and
generating the waveform-specific interval information based on a numerical value of ECG signal waveforms included in the ECG data.

5. The method of claim 4, wherein the generating of the waveform-specific interval information based on the numerical value of the ECG signal waveforms included in the ECG data includes:
extracting a feature value of each of the ECG signal waveforms included in the ECG data;
deriving a numerical value corresponding to the feature value of each of the ECG signal waveforms, and normalizing each of the ECG signal waveforms based on the derived numerical value; and
generating the waveform-specific interval information by combining the respective normalized ECG signal waveforms.

6. The method of claim 4, wherein the generating of the lead-specific integrated data includes:
generating the lead-specific integrated data by combining at least two of the ECG data, the gradient information, and the waveform-specific interval information.

7. The method of claim 1, wherein the generating of the N-dimensional input data includes:
generating a matrix form of 2D input data representing spatial information and time-series information of the ECG signal by arranging the lead-specific integrated data on a plane.

8. The method of claim 1, wherein the predicting of the chronic disease includes:
predicting a chronic disease of a subject corresponding to the ECG signal based on the N-dimensional input data by using the machine learning model.

9. The method of claim 8, wherein the machine learning model includes:
an encoder extracting a feature by receiving the N-dimensional input data; and
a decoder generating information on different types of chronic diseases based on the extracted feature.

10. The method of claim 8, wherein the machine learning model includes:
an encoder extracting a feature by receiving the N-dimensional input data; and
a decoder generating information on one chronic disease based on the extracted feature,
wherein when there are two or more decoders, the two or more decoders generate information on different types of chronic diseases respectively.

11. The method of claim 8, wherein the machine learning model is trained based on N-dimensional training data including spatial information and time-series information of the ECG signal.

12. The method of claim 8, wherein the generating of the prediction information on the chronic disease to be provided to the user includes:
generating a user interface based on prediction information on the chronic disease predicted through the machine learning model.

13. A computer program stored in a computer readable storage medium, wherein the computer program performs operations for predicting a chronic disease based on an ECG signal when executed by one or more processors, the operations comprising:
an operation of generating lead-specific integrated data based on the ECG signal, and generating N-dimensional input data based on the lead-specific integrated data;
an operation of predicting the chronic disease through a pre-trained machine learning model based on the N-dimensional input data; and
an operation of generating prediction information on the chronic disease to be provided to a user.

14. A computing device for predicting a chronic disease based on an ECG signal, comprising:
a processor including at least one core;
a memory including program codes executable in the processor; and
a network unit for receiving an ECG signal,
wherein the processor:
generates lead-specific integrated data based on the ECG signal, and generates N-dimensional input data based on the lead-specific integrated data;
predicts the chronic disease through a pre-trained machine learning model based on the N-dimensional input data; and
generates prediction information on the chronic disease to be provided to a user.

15. A user terminal, comprising:
a processor including at least one core;
a memory;
a network unit receiving analysis information of an ECG signal from a computing device; and
an output unit providing the analysis information of the ECG signal,
wherein the analysis information of the ECG signal includes prediction information on a chronic disease predicted based on the ECG signal, and
the prediction information on the chronic disease corresponds to information predicted through a pre-trained machine learning model, based on N-dimensional input data and lead-specific integrated data generated from the ECG signal.
